(19) United States
(12) Patent Application Publication
(10) Pub. No.: US 2012/0232508 A1
(43) Pub. Date: Sep. 13, 2012

(54) ABSORBENT ARTICLE

(76) Inventor: Makiko Urushihara, Mima-gun (JP)

(21) Appl. No.: 13/509,349

(22) PCT Filed: Nov. 2, 2010
(86) PCT No.: PCT/JP2010/006468

§ 371 Date: May 11, 2012

(30) Foreign Application Priority Data
Dec. 28, 2009 (JP) 2009-298705

(51) Int. Cl. A61L 15/22; A61L 15/58

(52) U.S. Cl. 604/365; 604/372

(57) ABSTRACT

An absorbent article comprising a top sheet, a back sheet and an absorbent laminate disposed between the top sheet and the back sheet, wherein: the absorbent laminate comprises a first absorbent layer and a second absorbent layer provided in this order from the top sheet side, and having a longitudinal direction and a width direction; the second absorbent layer contains an absorbent polymer and/or pulp fibers; the first absorbent layer contains an absorbent polymer but does not contain a pulp fiber between nonwoven fabric sheets; and the second absorbent layer extends outward in the longitudinal direction beyond the first absorbent layer.

### TECHNICAL FIELD

The present invention relates to an absorbent article such as an incontinence pad (including a light incontinence pad), a sanitary napkin, and a disposable diaper.

### BACKGROUND ART

Conventionally, there is known an absorbent article comprising a sheet-shaped absorbent body which contains an absorbent polymer but does not contain a pulp fiber between nonwoven fabric sheets. For example, Patent Literature 1 discloses an absorbent article comprising a laminate sheet in which two or more of the sheet-shaped absorbent bodies are laminated. Patent Literature 2 discloses an absorbent laminate comprising the sheet-shaped absorbent body and a fiber assembly layer which is disposed under the sheet-shaped absorbent body, that is a back sheet side, and which contains an absorbent polymer and pulp fibers.

### CITATION LIST

### Patent Literature

PTL 1: Japanese Patent Application Laid-Open Publication No. 2004-313580

PTL 2: Japanese Patent Application Laid-Open Publication No. 2004-275225

### SUMMARY OF INVENTION

### Technical Problem

The sheet-shaped absorbent body does not contain pulp fibers, and thus can be formed so as to be thin while maintaining its absorptive capacity. However, the absorption rate of an absorbent polymer is lower than that of pulp fibers, and therefore, when a bodily fluid such as urine is excreted in a large amount at one time, there is the possibility that the bodily fluid is not rapidly absorbed. Therefore, in the absorbent article in which the sheet-shaped absorbent body is provided on a top sheet side as disclosed in Patent Literatures 1 and 2, there is the possibility that a bodily fluid is not rapidly absorbed when a bodily fluid is excreted in a large amount at one time.

The present invention has been achieved in view of the above circumstances, and an object of the present invention is to provide an absorbent article which comprises the sheet-shaped absorbent body and can absorb a bodily fluid such as urine rapidly.

### Solution to Problem

An absorbent article of the present invention which solves the above problems comprises a top sheet, a back sheet and an absorbent laminate disposed between the top sheet and the back sheet, wherein: the absorbent laminate comprises a first absorbent layer and a second absorbent layer provided in this order from the top sheet side, and having a longitudinal direction and a width direction; the second absorbent layer contains an absorbent polymer and/or pulp fibers; the first absorbent layer contains an absorbent polymer but does not contain a pulp fiber between nonwoven fabric sheets; and the second absorbent layer extends outward in the longitudinal direction beyond the first absorbent layer. According to the absorbent article of the present invention, since the second absorbent layer extends outward in the longitudinal direction beyond the first absorbent layer, even when a bodily fluid is excreted in a large amount at one time and overflows from the edge of the first absorbent layer in the longitudinal direction, the bodily fluid is absorbed by the second absorbent layer, whereby the low absorption rate of the first absorbent layer is recovered. In particular, when the bodily fluid is excreted in the state where the wearer is in a prone position or in a supine position, the second absorbent layer which extends outward in the longitudinal direction beyond the first absorbent layer can effectively absorb the bodily fluid.

The second absorbent layer preferably contains an absorbent polymer but does not contain a pulp fiber between nonwoven fabric sheets. According to this constitution, the second absorbent layer can be also formed so as to be thin while having a high absorptive capacity. Therefore, the absorbent article which is formed slimly and has a high absorptive capacity is easily obtained.

It is preferable that the first absorbent layer has a plurality of absorbent polymer present regions, in each of which the absorbent polymer is provided, and an absorbent polymer absent region adjacent to the absorbent polymer present region between the nonwoven fabric sheets; the nonwoven fabric sheets of the first absorbent layer are joined together at the absorbent polymer absent region to form a sealing portion; and the absorbent polymer present regions of the first absorbent layer are disposed intermittently in the width direction of the absorbent laminate. According to this constitution, a bodily fluid is promoted to spread in the longitudinal direction on the surface of the first absorbent layer. Therefore, when a bodily fluid are excreted in a large amount at one time, the bodily fluid is easily absorbed by the second absorbent layer which extends outward in the longitudinal direction beyond the first absorbent layer.

It is also preferable that the second absorbent layer has a plurality of absorbent polymer present regions, in each of which the absorbent polymer is provided, and an absorbent polymer absent region adjacent to the absorbent polymer present region between the nonwoven fabric sheets; the nonwoven fabric sheets of the second absorbent layer are joined together at the absorbent polymer absent region to form a sealing portion; and the absorbent polymer present regions of the second absorbent layer are disposed intermittently in the width direction of the absorbent laminate. According to this constitution, a bodily fluid is promoted to spread in the longitudinal direction on the surface of the second absorbent layer. Therefore, a bodily fluid which has transferred from the edge, with respect to the longitudinal direction, of the first absorbent layer to the second absorbent layer easily moves toward the center of the second absorbent layer in the longitudinal direction, whereby the body fluid is less likely to overflow from the second absorbent layer.

The nonwoven fabric sheets of at least one of the first absorbent layer and the second absorbent layer may be partly heat-sealed at the absorbent polymer absent region. In this case, a bodily fluid easily spreads at a heat-sealed portion, which is formed by heat-sealing the first or second absorbent layer, in the planar direction of the first or second absorbent layer, whereas at a non-heat-sealed portion of the absorbent polymer absent region, a bodily fluid easily pass through the first or second absorbent layer in the thickness direction. Therefore, appropriate adjustment of the proportion of the heat-sealed portion and the non-heat-sealed portion allows optional adjustment of spread and permeation of a bodily fluid in the first or second absorbent layer.

Preferably, each of the absorbent polymer present regions is disposed in a shape of a practically straight line extending in the longitudinal direction and having a length of 75% or more of the absorbent laminate in the longitudinal direction; and the absorbent polymer present regions are aligned practically parallel each other in the width direction of the absorbent laminate. When the absorbent polymer present regions are provided in this manner, a bodily fluid easily spreads in the longitudinal direction in the first or second absorbent layer, and further, the absorptive capacity of the first or second absorbent layer is easily ensured since the absorbent polymer present regions are disposed so as to have relatively large areas.

Preferably, a maximum distance between the adjacent absorbent polymer present regions of the first absorbent layer is larger than that of the second absorbent layer. In this case, permeation of a bodily fluid in the first absorbent layer is enhanced rather than that in the second absorbent layer, and therefore, the absorbent laminate can absorb a bodily fluid more rapidly.

At the sealing portion, the nonwoven fabric sheets of the first absorbent layer or the second absorbent layer is preferably kept joined together when the first absorbent layer or the second absorbent layer absorbs a body fluid. When the sealing portion is maintained even in the case where the absorbent polymer provided in the first absorbent layer or the second absorbent layer absorbs a body fluid to swell, permeation of a bodily fluid in the first absorbent layer or the second absorbent layer is easily ensured.

Preferably, an adhesive is applied to the nonwoven fabric sheet to form an adhesive layer; the absorbent polymer disposed at the absorbent polymer present region is fixed to the nonwoven fabric sheet by the adhesive layer; and the nonwoven fabric sheets are joined together at the absorbent polymer absent region by the adhesive layer. According to this constitution, the absorbent polymer is less likely to move in the first absorbent layer and/or the second absorbent layer, and the absorbent capability of the first absorbent layer and/or the second absorbent layer is sufficiently ensured. In addition, a feeling of discomfort due to unevenly location of the absorbent polymer is less likely to be provided to a wearer.

### Advantageous Effects of Invention

The absorbent article of the present invention can rapidly absorb a bodily fluid such as urine.

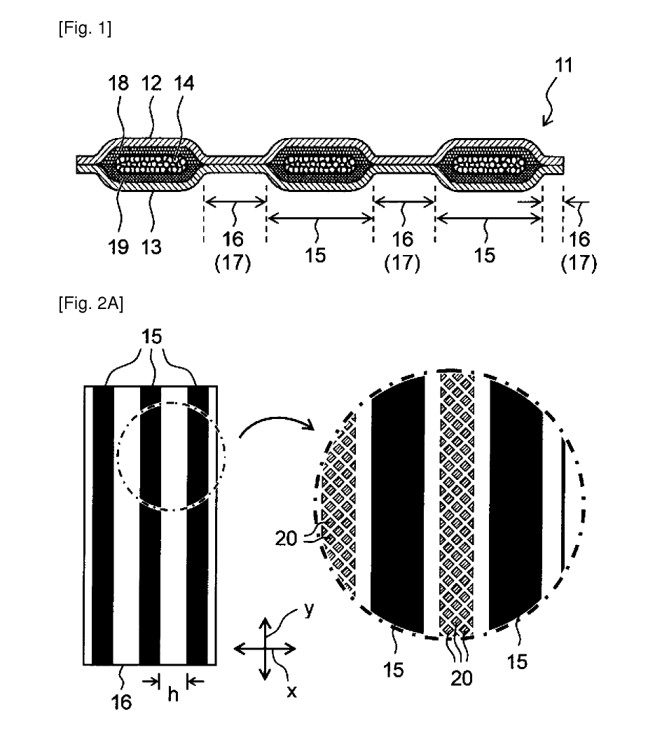

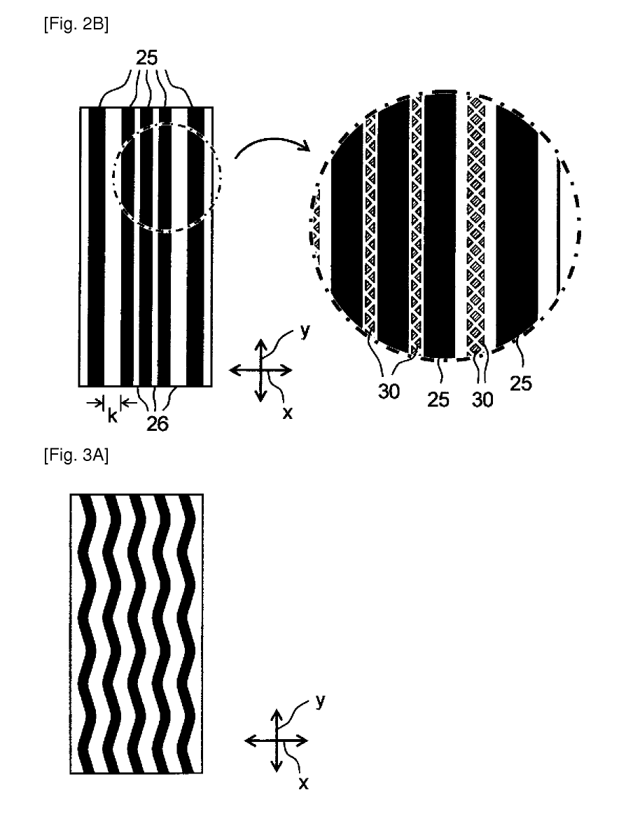

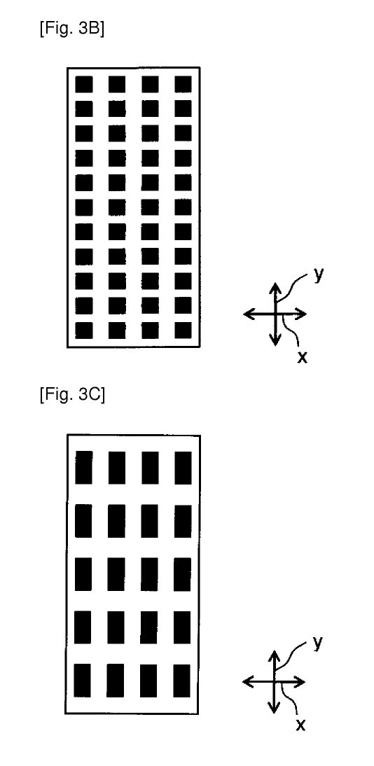

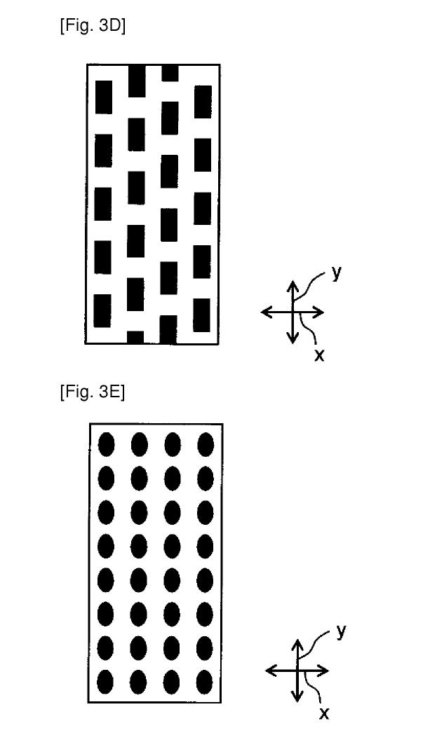

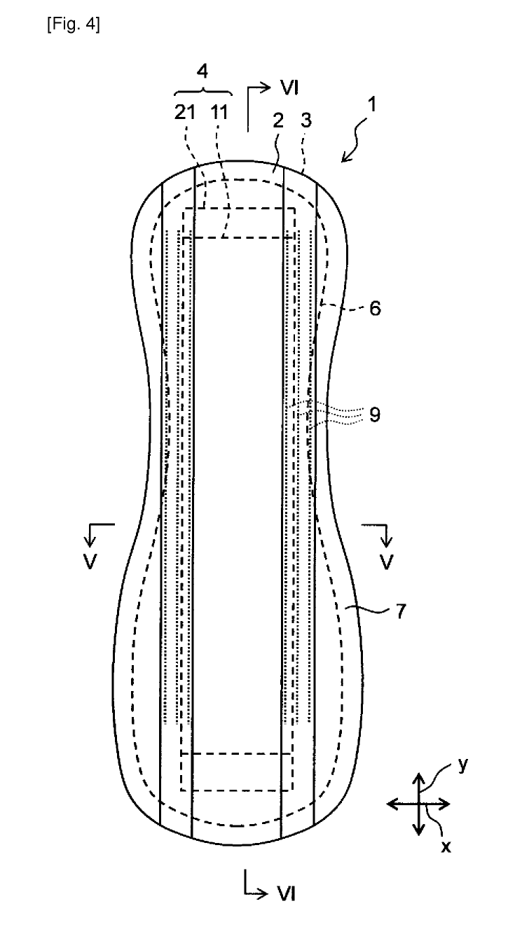

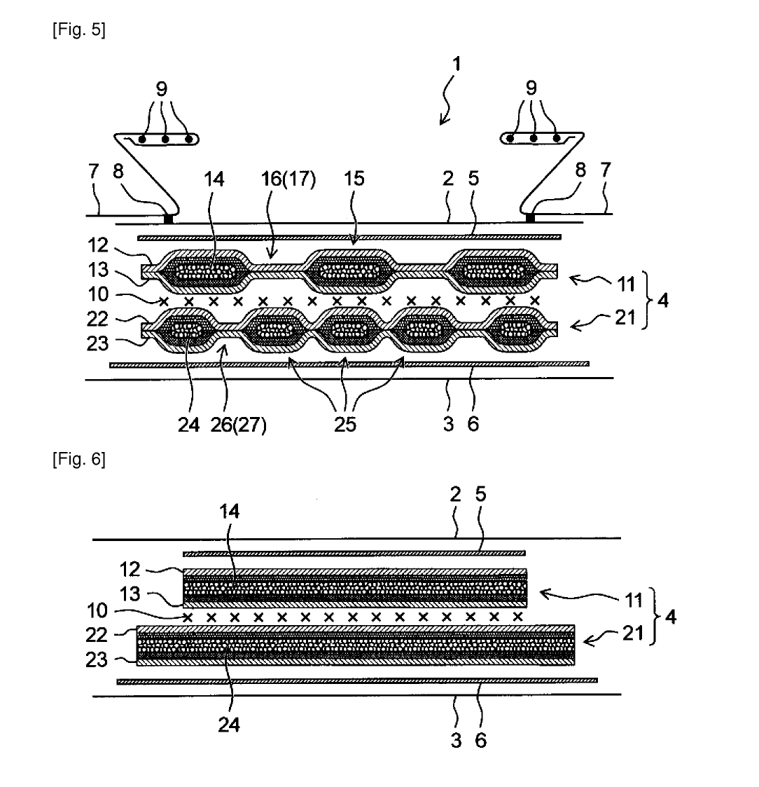

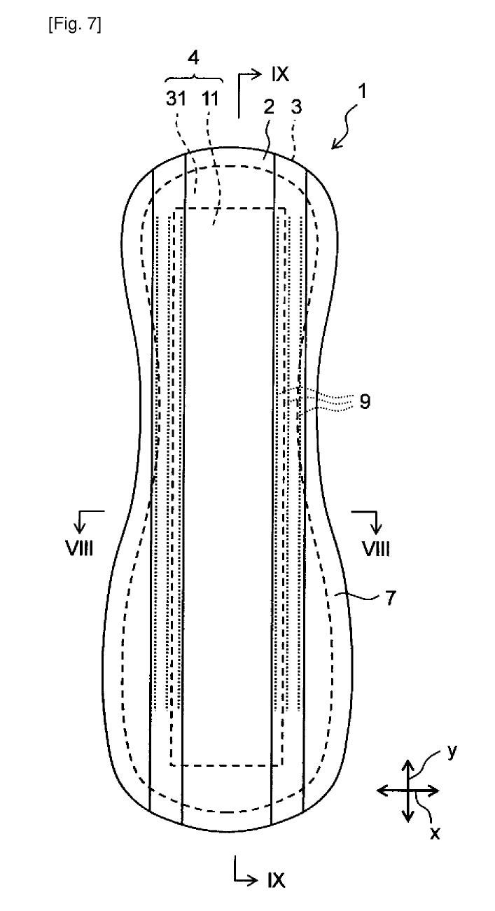

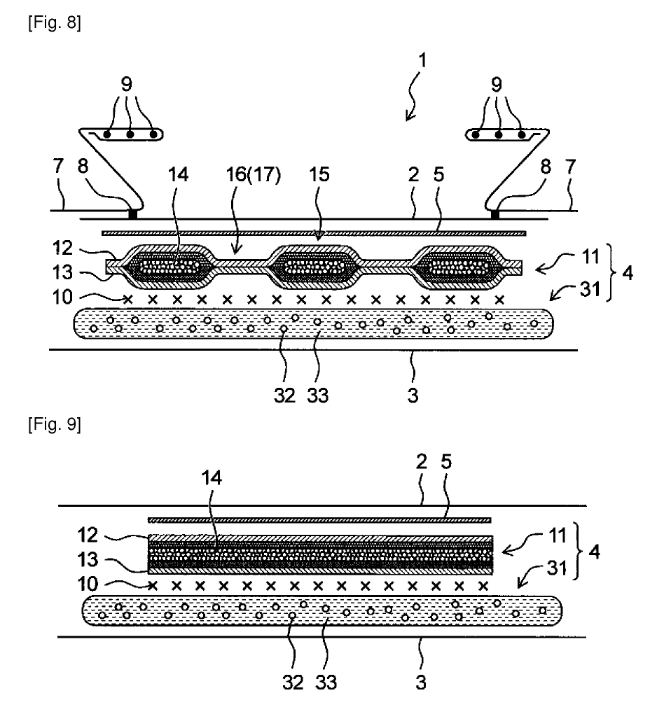

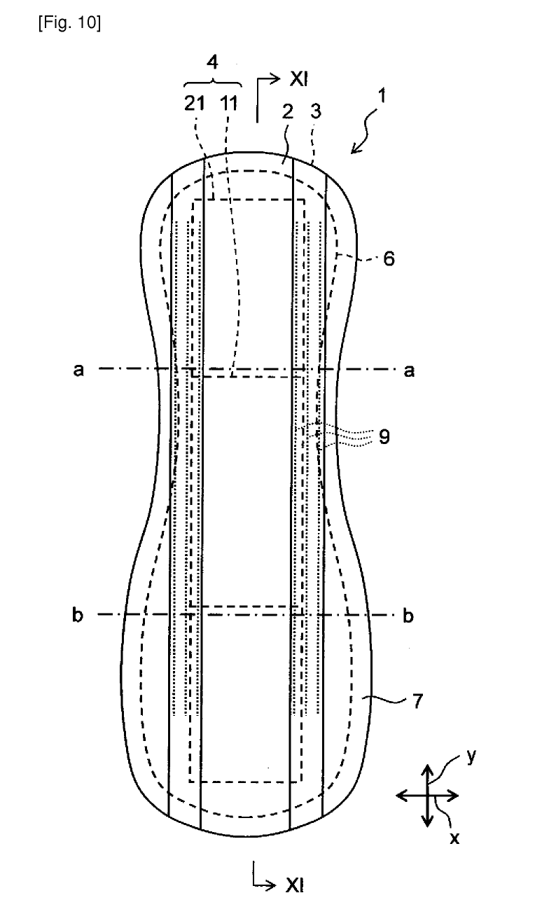

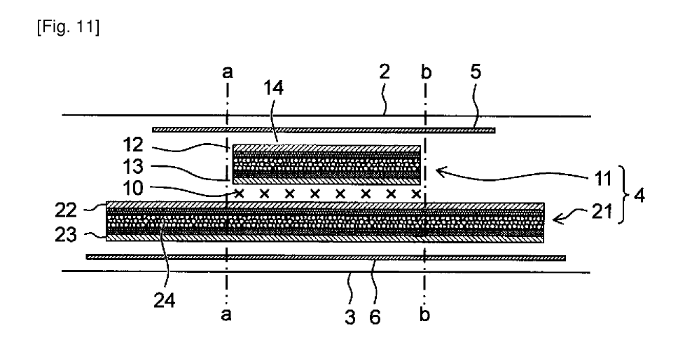

### BRIEF DESCRIPTION OF DRAWINGS

[FIG. 1] FIG. 1 shows an example of a cross-sectional view of a first absorbent layer.

[FIG. 2A] FIG. 2A shows an example of an arrangement pattern of an absorbent polymer present region and an example of a sheet-shaped absorbent layer provided with heat-sealed portions.

[FIG. 2B] FIG. 2B shows an example of an arrangement pattern of the absorbent polymer present region and an example of the sheet-shaped absorbent layer provided with heat-sealed portions.

[FIG. 3A] FIG. 3A shows an example of an arrangement pattern of the absorbent polymer present region.

[FIG. 3B] FIG. 3B shows an example of an arrangement pattern of the absorbent polymer present region.

[FIG. 3C] FIG. 3C shows an example of an arrangement pattern of the absorbent polymer present region.

[FIG. 3D] FIG. 3D shows an example of an arrangement pattern of the absorbent polymer present region.

[FIG. 3E] FIG. 3E shows an example of an arrangement pattern of the absorbent polymer present region.

[FIG. 4] FIG. 4 shows a plan view of an absorbent article in accordance with an embodiment of the present invention.

[FIG. 5] FIG. 5 shows a cross-sectional view taken along line V-V of the absorbent article shown in FIG. 4.

[FIG. 6] FIG. 6 shows a cross-sectional view taken along line VI-VI of the absorbent article shown in FIG. 4.

[FIG. 7] FIG. 7 shows a plan view of an absorbent article in accordance with another embodiment of the present invention.

[FIG. 8] FIG. 8 shows a cross-sectional view taken along line VIII-VIII of the absorbent article shown in FIG. 7.

[FIG. 9] FIG. 9 shows a cross-sectional view taken along line IX-IX of the absorbent article shown in FIG. 7.

[FIG. 10] FIG. 10 shows a plan view of an absorbent article in accordance with still another embodiment of the present invention.

[FIG. 11] FIG. 11 shows a cross-sectional view taken along line XI-XI of the absorbent article shown in FIG. 10.

### DESCRIPTION OF EMBODIMENTS

An absorbent article of the present invention comprises a top sheet, a back sheet and an absorbent laminate disposed between the top sheet and the back sheet. The top sheet is a sheet which is located on a wearer's side, that is an inner side, when the absorbent article is worn. The material of the top sheet is not restricted as long as it is liquid-permeable. The back sheet is a sheet which is located on an opposite side of a wearer, that is an outer side, when the absorbent article is worn. The material of the back sheet is not restricted as long as it is liquid-impermeable.

The top sheet and the back sheet may be composed of a nonwoven fabric, a woven fabric, a knitted fabric, a plastic film, a laminate of a plastic film and a nonwoven fabric, or the like. Examples of the laminate include a laminate in which a sheet of a nonwoven fabric and a sheet of a plastic film are stacked, and a laminate in which a plastic film is interposed between nonwoven fabrics. When a plastic film or a laminate including a plastic film is used for the top sheet, it is preferable that pores for allowing liquid to permeate through are formed in the plastic film. The top sheet is preferably made of a nonwoven fabric. The back sheet is preferably made of a nonwoven fabric or a plastic film.

When a nonwoven fabric is used for the top sheet or the back sheet, a nonwoven fabric manufactured by a spunbonding method, an air-through method, a point bonding method, a melt blowing method, an airlaid method, a combination of these methods, or the like, is preferably used. Also, a nonwoven fabric manufactured by an SMS method which is a combination of the spunbonding method and the melt blowing method may be used.

When a nonwoven fabric is used for the top sheet or the back sheet, a material of the nonwoven fabric can be selected as appropriate from synthetic fibers such as polypropylene, polyethylene, polyester (e.g., PET) and polyamide; natural fibers such as pulp and silk. Also, composite fibers can be used as the synthetic fibers. Among them, polypropylene, polyethylene, PET, or composite fibers obtained by combining these materials, are preferred. When such a nonwoven fabric is used, a sheet having a high strength and excellent texture is easily obtained.

The absorbent laminate is disposed between the top sheet and the back sheet, and absorbs excrement such as urine. The absorbent laminate comprises a first absorbent layer and a second absorbent layer provided in this order from the top sheet side. Preferably, the first absorbent layer is provided adjacent to the second absorbent layer. The first absorbent layer is provided so as to be closer to the top sheet than the second absorbent layer. Therefore, the first absorbent layer receives a bodily fluid such as urine, which has passed through the top sheet to reach the absorbent laminate, earlier than the second absorbent layer, basically. The second absorbent layer receives the bodily fluid which has passed through the first absorbent layer or which has overflowed from the first absorbent layer, basically.

The absorbent laminate has a longitudinal direction and a width direction. The longitudinal direction means a direction extending in a front-back direction at a crotch of a wearer when the wearer wears the absorbent article. With respect to the longitudinal direction, an abdomen side of a wearer is referred to as a front side, and a buttocks side of the wearer is referred to as a rear side. The width direction means a direction orthogonal to the longitudinal direction on the same plane as the absorbent laminate. A direction on the plane formed by the longitudinal direction and the width direction is defined as a planar direction. In addition, in the present invention, upper side means a wearer's side when the absorbent article is worn, and lower side means the side opposite to a wearer when the absorbent article is worn, that is an outer side. In addition, the direction from the upper side to the lower side is referred to as a thickness direction.

The shape (planar shape) of the absorbent laminate is not particularly limited. The shapes (planar shapes) of the first absorbent layer and the second absorbent layer are not also particularly limited. The shapes of the first absorbent layer and the second absorbent layer may be same or different from each other. The shapes of the absorbent laminate, the first absorbent layer and the second absorbent layers are determined as appropriate according to application, and examples of the shape of the absorbent laminate include, for example, a rectangular shape, an hourglass shape, a center nipped-in gourd shape, and a battledore shape.

The first absorbent layer contains an absorbent polymer but does not contain a pulp fiber between nonwoven fabric sheets. Thus, the first absorbent layer is formed by providing an absorbent polymer between nonwoven fabric sheets, and a pulp fiber is not provided between the nonwoven fabric sheets. Since the first absorbent layer contains an absorbent polymer but does not contain a pulp fiber, the first absorbent layer can be formed so as to be thin while having a high absorptive capacity. Hereinafter, an object which contains an absorbent polymer but does not contain a pulp fiber between nonwoven fabric sheets may be referred to as a sheet-shaped absorbent body.

Examples of the absorbent polymer provided in the first absorbent layer include polyacrylic acid-based absorbent polymers such as sodium polyacrylate; starch-based absorbent polymers such as a starch-acrylonitrile graft copolymer, a starch-acrylic acid graft copolymer, and a starch-acrylamide graft copolymer; and polyvinyl alcohol-based absorbent polymers such as a crosslinked polyvinyl alcohol. As the absorbent polymer, polyacrylic acid-based absorbent polymers such as sodium polyacrylate are preferably used, since they have a high absorptive capacity.

The nonwoven fabric sheets used in the first absorbent layer are liquid-permeable. For such nonwoven fabric sheets, for example, hydrophilic fibers such as cellulose, rayon, and cotton; and hydrophilized hydrophobic fibers such as polypropylene, polyethylene, polyester, and polyamide with a surfactant may be used. When the nonwoven fabric sheets are partly heat-sealed as described below, hydrophilized hydrophobic fibers, such as polypropylene, polyethylene, polyester, and polyamide, with a surfactant are preferably used for the nonwoven fabric sheets, since heat-sealing thereof is facilitated.

In the first absorbent layer, the absorbent polymer may be provided between two nonwoven fabric sheets, that are an upper nonwoven fabric sheet on the top sheet side and a lower nonwoven fabric sheet on the back sheet side. In this case, each of the upper nonwoven fabric sheet and the lower nonwoven fabric sheet may be formed of one nonwoven fabric sheet, or may be formed by two or more nonwoven fabric sheets being laminated on each other. Alternatively, concerning the upper nonwoven fabric sheet and the lower nonwoven fabric sheet, one nonwoven fabric sheet may be folded at a fold line to define two portions across the fold line, one of the two portions may serve as the upper nonwoven fabric sheet, and the other of the two portions may serve as the lower nonwoven fabric sheet. In this case, the absorbent polymer is provided inside the folded one nonwoven fabric sheet.

The second absorbent layer contains an absorbent polymer and/or pulp fibers. As the second absorbent layer, an object which contains an absorbent polymer but does not contain a pulp fiber between nonwoven fabric sheets, that is the sheet-shaped absorbent body, may be used, for example. Alternatively, a shaped product of pulp fibers that is formed into a predefined shape (a shaped product containing pulp fibers); a shaped product, in which an absorbent polymer is dispersed in pulp fibers, that is formed into a predefined shape (a shaped product containing an absorbent polymer and pulp fibers); a wrapped shaped product in which the shaped product is wrapped with a covering sheet such as a paper (e.g., tissue paper) and a liquid-permeable nonwoven fabric; may be used as the second absorbent layer.

In the case that the second absorbent layer contains an absorbent polymer, an absorbent polymer which can be used in the first absorbent layer may be used for the second absorbent layer.

In the case that the second absorbent layer contains pulp fibers, split pulp fibers are preferably used as the pulp fibers. In addition, the split pulp fibers are preferably used in a form of a fiber mass. When the second absorbent layer contains pulp fibers, the second absorbent layer can rapidly absorb a body fluid such as urine. Pulp fibers may reversibly hold a body fluid, not absorbing and irreversibly fixing a body fluid like an absorbent polymer; however, in the present description, the term “absorb” includes the meaning of “hold”.

In the absorbent article of the present invention, the second absorbent layer extends outward in the longitudinal direction beyond the first absorbent layer. In other word, the longitudinal edge, that is the edge in the longitudinal direction, of the second absorbent layer locates outside the longitudinal edge of the first absorbent layer. It is sufficient that at least one of the longitudinal edges of the second absorbent layer locates outside the first absorbent layer in the longitudinal direction. Preferably, the front edge of the second absorbent layer, that is the edge of the front side of the second absorbent layer, locates outside the first absorbent layer in the longitudinal direction. More preferably, both longitudinal edges of the second absorbent layer locate outside the first absorbent layer in the longitudinal direction.

In the first absorbent layer, which contains the absorbent polymer between the nonwoven fabrics and does not contain pulp fibers therebetween, there is the possibility that a body fluid is not rapidly absorbed by the first absorbent layer and a part of the body fluid overflows from the surface of the first absorbent layer when a bodily fluid such as urine is excreted in a large amount at one time, because the absorption rate of the absorbent polymer is lower than that of the pulp fibers. In particular, when the wearer is in a prone position or in a supine position, there is the possibility that a body fluid such as urine diffuses in the first absorbent layer in the longitudinal direction and overflows from the first absorbent layer. However, in the absorbent article of the present invention, since the second absorbent layer extends beyond the first absorbent layer in the longitudinal direction, even when the wearer is in a prone position or in a supine position, a body fluid which has overflowed from the longitudinal edge of the first absorbent layer is absorbed by the second absorbent layer, and therefore, the low absorption rate of the first absorbent layer is recovered. In particular, when it is taken into consideration that the urethral orifice of a wearer is positioned at the front side of the crotch part, at least the front edge of the second absorbent layer preferably locates outside the first absorbent layer.

The absorbent article of the present invention is also excellent in preventing return (wet back) of a bodily fluid to a skin side. Since the first absorbent layer contains the absorbent polymer but does not contain a pulp fiber between the nonwoven fabric sheets, when the first absorbent layer absorbs a bodily fluid, the surface thereof is kept relatively dry. In addition, even when a part of the bodily fluid absorbed by the second absorbent layer is released, the bodily fluid released from the second absorbent layer is easily prevented from reaching the skin of a wearer due to the presence of the first absorbent layer provided so as to be closer to the top sheet than the second absorbent layer. Therefore, the skin of a wearer is kept clean, thereby improving a feel of wearing.

The lengths, with respect to the width direction of the absorbent laminate, of the first absorbent layer and the second absorbent layer are not limited, however, it is preferable that the edge of the second absorbent layer in the width direction coincide with the edge of the first absorbent layer in the width direction or locates outside the edge of the first absorbent layer in the width direction. In particular, when the edge of the second absorbent layer in the width direction locates outside the edge of the first absorbent layer in the width direction, a bodily fluid which has overflowed from the edge of the first absorbent layer in the width direction can be also absorbed by the second absorbent layer.

The relation of the length in the longitudinal direction between the first absorbent layer and the second absorbent layer is not restricted as long as the second absorbent layer extends outward in the longitudinal direction beyond the first absorbent layer. For example, in the case that the absorbent article is folded at one or more fold line(s) extending in the width direction, thereby folded in two or more in the longitudinal direction, the first absorbent layer is preferably provided so as not to cross at least one of the one or more fold line(s). When the first absorbent layer is provided in this manner, the folded absorbent article obtained by folding the absorbent article compactly can be formed slimly. In the case that the absorbent article is folded at two fold lines extending in the width direction, thereby folded in three in the longitudinal direction, the first absorbent layer may be disposed, for example, so as to exist between the two fold lines and not to cross at least one of the two fold lines. In the case that the absorbent article is folded at three fold lines extending in the width direction, thereby folded in four in the longitudinal direction, the first absorbent layer may be disposed, for example, so as to cross the middle fold line and not to cross at least one of the side fold lines.

In order that a bodily fluid such as urine is promoted to spread in the longitudinal direction in the first absorbent layer, the absorbent polymers are preferably disposed intermittently in the width direction. Thus, it is preferable that the first absorbent layer has a plurality of absorbent polymer present regions, in each of which the absorbent polymer is provided, and an absorbent polymer absent region adjacent to the absorbent polymer present region between the nonwoven fabric sheets; the nonwoven fabric sheets of the first absorbent layer are joined together at the absorbent polymer absent region to form a sealing portion; and the absorbent polymer present regions of the first absorbent layer are disposed intermittently in the width direction of the absorbent laminate.

When the absorbent polymer absent region is provided in the first absorbent layer, and the nonwoven fabric sheets are joined together at this region to form the sealing portion, a bodily fluid easily pass through the absorbent polymer absent region, and easily spreads on the surface of the first absorbent layer at the absorbent polymer absent region. Further, when the absorbent polymer present regions are disposed intermittently in the width direction, a bodily fluid is promoted to spread in the longitudinal direction on the surface of the first absorbent layer. Therefore, when a bodily fluid are excreted in a large amount at one time to overflow from the surface of the first absorbent body, the bodily fluid is easily absorbed by the second absorbent layer which extends outward in the longitudinal direction beyond the first absorbent layer.

The second absorbent layer preferably contains an absorbent polymer but does not contain a pulp fiber between nonwoven fabric sheets. That is, the second absorbent layer is preferably composed of the sheet-shaped absorbent body. When the second absorbent layer is composed of the sheet-shaped absorbent body as well as the first absorbent layer, the second absorbent layer can be also formed so as to be thin while having a high absorptive capacity. Therefore, the absorbent article which is formed slimly and has a high absorptive capacity is easily obtained.

In the case that the second absorbent layer is composed of the sheet-shaped absorbent body, an absorbent polymer and a nonwoven fabric sheet which can be used in the first absorbent layer may be used for the second absorbent layer.

In the case that the second absorbent layer is composed of the sheet-shaped absorbent body, it is preferable that the second absorbent layer has a plurality of absorbent polymer present regions, in each of which the absorbent polymer is provided, and an absorbent polymer absent region adjacent to the absorbent polymer present region between the nonwoven fabric sheets; the nonwoven fabric sheets of the second absorbent layer are joined together at the absorbent polymer absent region to form a sealing portion; and the absorbent polymer present regions of the second absorbent layer are disposed intermittently in the width direction of the absorbent laminate. In this case, a part of the bodily fluid easily permeate the absorbent polymer absent region of the second absorbent layer to reach the lower side (the back sheet side) of the second absorbent layer, and therefore, the bodily fluid is easily absorbed from both the upper and lower sides of the second absorbent layer, whereby rapid absorption of a bodily fluid by the second absorbent layer is achieved. In addition, a bodily fluid which has transferred to the second absorbent layer easily spreads on the surface of the second absorbent layer at the absorbent polymer absent region. Further, since the absorbent polymer present regions of the second absorbent layer are disposed intermittently in the width direction of the absorbent laminate, a bodily fluid is promoted to spread in the longitudinal direction on the surface of the second absorbent layer. Therefore, a bodily fluid which has transferred from the longitudinal edge of the first absorbent layer to the second absorbent layer easily moves toward the center of the second absorbent layer in the longitudinal direction, whereby the body fluid is less likely to overflow from the second absorbent layer.

The sealing portion formed in the sheet-shaped absorbent body may be formed by joining the nonwoven fabric sheets together by an adhesive or heat-sealing (thermal fusion-bonding). The sealing portion also may be formed by ultrasonic-bonding the nonwoven fabric sheets together.

At the sealing portion, the nonwoven fabric sheets are preferably kept joined together when the first absorbent layer or the second absorbent layer absorbs a bodily fluid. That is, in the first absorbent layer, the nonwoven fabric sheets of the first absorbent layer are preferably kept joined together at the sealing portion when the first absorbent layer absorbs a bodily fluid. In the second absorbent layer, if the second absorbent layer is composed of the sheet-shaped absorbent body, the nonwoven fabric sheets of the second absorbent layer are preferably kept joined together at the sealing portion when the second absorbent layer absorbs a bodily fluid. When the sheet-shaped absorbent body absorbs a bodily fluid, the absorbent polymer provided between the nonwoven fabric sheets swells, and therefore, the joining of the nonwoven fabric sheets at the sealing portion may possibly separate. In this case, the spread of a bodily fluid at the absorbent polymer absent region may be inhibited, and it may become difficult for a bodily fluid to pass through the absorbent polymer absent region. Therefore, the nonwoven fabric sheets of the sheet-shaped absorbent body are preferably kept joined together when the sheet-shaped absorbent body absorbs.

In order that the joining of the nonwoven fabric sheets is maintained when the sheet-shaped absorbent body absorbs a bodily fluid, it is preferable that the upper limit of the absorbent polymer content in the absorbent polymer present region is defined. Thus, the content of the absorbent polymer in the absorbent polymer present region is preferably 400 g/m² or less, and more preferably 385 g/m² or less. Meanwhile, in order to ensure sufficient absorption amount in the absorbent polymer present region of the sheet-shaped absorbent body, the content of the absorbent polymer in the absorbent polymer present region is preferably 100 g/m² or more, and more preferably 150 g/m² or more.

In order that the joining of the nonwoven fabric sheets is maintained when the sheet-shaped absorbent body absorbs a bodily fluid, it is also preferable that the nonwoven fabric sheets are adhered together by a rubber adhesive or a styrene based-elastomer, or heat-sealed together.

It is preferable that an adhesive is applied to the nonwoven fabric sheet of the first absorbent layer and/or the second absorbent layer to form an adhesive layer, and the absorbent polymer disposed at the absorbent polymer present region is fixed to the nonwoven fabric sheet by the adhesive layer. That is, it is preferable that an adhesive is applied to the nonwoven fabric sheet of the first absorbent layer to form an adhesive layer, and the absorbent polymer disposed at the absorbent polymer present region is fixed to the nonwoven fabric sheet by the adhesive layer. In the case that the second absorbent layer is composed of the sheet-shaped absorbent body, it is preferable that an adhesive is applied to the nonwoven fabric sheet of the second absorbent layer to form an adhesive layer, and the absorbent polymer disposed at the absorbent polymer present region is fixed to the nonwoven fabric sheet by the adhesive layer. More preferably, the second absorbent layer is composed of the sheet-shaped absorbent body, and an adhesive is applied to the nonwoven fabric sheets of both the first absorbent layer and the second absorbent layer to form adhesive layers, and the absorbent polymer disposed at the absorbent polymer present region is fixed to the nonwoven fabric sheet by the adhesive layer.

The adhesive layer may be disposed on at least one of the nonwoven fabric sheets by which the absorbent polymer is sandwiched; and preferably, the adhesive layers are disposed on both of the nonwoven fabric sheets by which the absorbent polymer is sandwiched. Here, in the absorbent polymer present region, it is sufficient that at least a part of the absorbent polymer provided is fixed to the adhesive layer, and for example, the absorbent polymer in contact with the adhesive layer may be fixed to the adhesive layer. When the absorbent polymer is fixed to the nonwoven fabric sheets by the adhesive layer, the absorbent polymer is less likely to move in the sheet-shaped absorbent body prior to the absorbent polymer absorbing a bodily fluid, and the absorbent capability of the sheet-shaped absorbent body is sufficiently ensured. In addition, the absorbent polymer is less likely to be unevenly located in the sheet-shaped absorbent body since the absorbent polymer is suppressed to move therein, and thus a feeling of discomfort is less likely to be provided to a wearer. Even after the absorbent polymer absorbs a bodily fluid, the gelled absorbent polymer is less likely to move in the sheet-shaped absorbent body, and as a result, the absorbent polymer is less likely to form a lump to provide a feeling of discomfort to a wearer.

At the absorbent polymer absent region, the nonwoven fabric sheets are preferably joined together by the adhesive layer. In this case, the joining of the nonwoven fabric sheets, that is the sealing portion, is easily maintained, even when the absorbent polymer disposed at the absorbent polymer present region absorbs a bodily fluid to swell.

It is preferred that the adhesive layer does not inhibit absorption and swelling of the absorbent polymer while fixing the absorbent polymer at the absorbent polymer present region. In this respect, the adhesive layer is preferably formed into a net-like structure.

As a method of forming the adhesive layer into a net-like structure, a method for discharging a molten adhesive from a plurality of nozzles in a thread form (a curtain spray method, a spiral coating method or an omega coating method) may be used. In the curtain spray method, for example, a curtain spray coater which comprises: a plurality of small discharge holes arranged linearly; and air injection ports, which are capable of injecting hot air at high speed, provided in the vicinity of each of the discharge holes; may be used. Air is blown off to a molten adhesive discharged from the each discharging hole in a thread form, whereby the adhesive can be applied to a nonwoven fabric as an assembly of nets in which many filamentous adhesives randomly adhere to one another. In the spiral coating method, for example, a spiral spray nozzle gun, in which three or more air injection ports capable of blowing out air in a direction of a center of the nozzle are provided point symmetrically in the vicinity of a hot-melt adhesive discharging hole, may be used. By using the spiral spray nozzle gun, an adhesive layer in which an adhesive filament is formed into a spiral form can be applied to a nonwoven fabric. In the omega coating method, for example, an adhesive layer having an omega-shaped pattern is formed by, while continuously discharging an adhesive from a coating head located above a nonwoven fabric, moving linearly the nonwoven fabric relative to the coating head, and changing the dropping direction of the adhesive by air blow or the like so as to reciprocate substantially perpendicularly to the moving direction of the nonwoven fabric.

As a method of forming the adhesive layer into a net-like structure, it may be employed that a hot-melt adhesive is fell from an adhesive discharging hole in a thread form having a square cross-sectional shape, and applied to a nonwoven fabric in the state that the dropping hot-melt adhesive is laterally waved by applying slit air, which is provided in an acute angle direction relative to the adhesive discharging hole, to the front and back sides of the dropping hot-melt adhesive. According to this method, an adhesive layer having a zig-zag pattern or a meandering pattern is formed on the nonwoven fabric. Alternatively, by a coater method, an adhesive may be applied to a nonwoven fabric in a very thin stripe shape to form the adhesive layer, and the absorbent polymer may be fixed thereto. According to this method as well, the same effect is obtained as in the case of forming the adhesive layer into a net-like structure.

Examples of the adhesive used for the adhesive layer include, for example, rubber adhesives such as natural rubbers, butyl rubbers and polyisoprene; styrene elastomers such as styrene-isoprene-styrene block copolymer (SIS), styrene-butadiene-styrene block copolymer (SBS), styrene-ethylene-butadiene-styrene block copolymer (SEBS), and styrene-ethylene-propylene-styrene block copolymer (SEPS); ethylene-vinyl acetate copolymer (EVA); polyester; acryl elastomers; and polyolefin elastomers. These exemplified adhesives may be used either alone or as a combination of at least two of them. It is preferable that the adhesive has such an adhesive force that the absorbent polymer can be prevented from falling off after absorbing a bodily fluid, and is stretchable to such an extent as to allow swelling of the absorbent polymer. It is also preferable that the adhesive has such an adhesive force that the nonwoven fabric sheets are kept joined together even when the absorbent polymer absorbs a bodily fluid and swells. In these respects, rubber adhesives and styrene elastomers are preferably used.

FIG. 1 shows an example of a cross-sectional view of the first absorbent layer provided with the adhesive layers. A first absorbent layer 11 comprises an upper first nonwoven fabric sheet 12, a lower first nonwoven fabric sheet 13, an upper first adhesive layer 18 formed by applying an adhesive to the upper first nonwoven fabric sheet 12, a lower first adhesive layer 19 formed by applying an adhesive to the lower first nonwoven fabric sheet 13, and absorbent polymers 14 disposed between the upper first adhesive layer 18 and the lower first adhesive layer 19. The first absorbent layer 11 has a plurality of absorbent polymer present regions 15 and absorbent polymer absent regions 16 adjacent to the absorbent polymer present region 15. Absorbent polymers 14 of the absorbent polymer present regions 15 are fixed to the upper first nonwoven fabric sheet 12 and the lower first nonwoven fabric sheet 13 by the upper first adhesive layer 18 and the lower first adhesive layer 19, respectively. At the absorbent polymer absent regions 16, the upper first nonwoven fabric sheet 12 and the lower first nonwoven fabric sheet 13 are joined together by the upper first adhesive layer 18 and the lower first adhesive layer 19 to form sealing portions 17. In the above, the first absorbent layer is explained with reference to FIG. 1, as an example. In the case that the sheet-shaped absorbent body is used as the second absorbent layer, the word “first” in the above description concerning FIG. 1 is replaced by “second”.

It is preferable that the nonwoven fabric sheets of at least one of the first absorbent layer and the second absorbent layer are partly heat-sealed at the absorbent polymer absent region. That is, the nonwoven fabric sheets of the first absorbent layer are preferably partly heat-sealed at the absorbent polymer absent region. In the case that the second absorbent layer is composed of the sheet-shaped absorbent body, the nonwoven fabric sheets of the second absorbent layer are preferably partly heat-sealed at the absorbent polymer absent region.

When the nonwoven fabric sheets are partly heat-sealed at the absorbent polymer absent region, a bodily fluid easily spreads at the heat-sealed portion in the planar direction of the sheet-shaped absorbent body. Meanwhile, at a non-heat-sealed portion of the absorbent polymer absent region, a bodily fluid easily passes through the sheet-shaped absorbent body in the thickness direction. Therefore, appropriate adjustment of the proportion of the heat-sealed portion and the non-heat-sealed portion allows optional adjustment of the spread and permeation of a bodily fluid in the sheet-shaped absorbent body. Partial heat-sealing may be achieved by heat-sealing the nonwoven fabric sheets in a predefined pattern.

The nonwoven fabric sheets may be joined together by the adhesive layer at the absorbent polymer absent region, at which the nonwoven fabric sheets may be further partly heat-sealed. Alternatively, the adhesive layer may not be provided at the absorbent polymer absent region, at which the nonwoven fabric sheets may be partly heat-sealed. Still alternatively, the nonwoven fabric sheets may not be partly heat-sealed at the absorbent polymer absent region, at which the nonwoven fabric sheets may be joined together by the adhesive layer.

In the case that the sheet-shaped absorbent body has the absorbent polymer present region and the absorbent polymer absent region, the absorbent polymer present regions are preferably disposed intermittently in the width direction of the absorbent laminate. This is explained referring to FIGS. 2A, 2B and 3A to 3E, which show examples of an arrangement pattern of the absorbent polymer present region. In FIGS. 2A, 2B and 3A to 3E, the absorbent polymer present region is expressed in black and the absorbent polymer absent region is expressed in white. In the drawings, an arrow x represents the width direction of the absorbent laminate and an arrow y represents the longitudinal direction of the absorbent laminate.

In FIGS. 2A, 2B, and 3A to 3E, a plurality of the absorbent polymer present regions, in each of which the absorbent polymer is provided, and the absorbent polymer absent region adjacent to the absorbent polymer present region are provided; and the absorbent polymer present regions are disposed intermittently in the width direction x of the absorbent laminate.

In FIGS. 2A and 2B, each of the absorbent polymer present regions is disposed in the shape of a practically straight line extending in the longitudinal direction y of the absorbent laminate and having a length approximately equal to the length of the sheet-shaped absorbent body in the longitudinal direction y. When the absorbent polymer present regions are disposed in these manners, a bodily fluid easily spreads in the longitudinal direction y, and further, since the absorbent polymer present regions are disposed so as to have relatively large areas in total (e.g., when compared to FIGS. 3B to 3E which are described below), the absorptive capacity of the sheet-shaped absorbent body is easily enhanced. The widths of the plurality of absorbent polymer present regions may be same or different from each other. In addition, the plurality of absorbent polymer present regions may be disposed at regular intervals, or may not be disposed at regular intervals. For example, in FIG. 2A, the plurality of absorbent polymer present regions have the substantially same width, and are disposed at substantially regular intervals. On the other hand, in FIG. 2B, the absorbent polymer present regions in a middle portion in the width direction x are disposed at short intervals so as to have narrow widths, and the absorbent polymer present regions in side portions in the width direction x are disposed at long intervals so as to have wide widths.

In FIGS. 2A and 2B, the respective absorbent polymer present regions are disposed in the shape of straight lines extending in the longitudinal direction y; however, the absorbent polymer present regions may be disposed in the shape of meandering lines extending in the longitudinal direction y as shown in FIG. 3A. Further, the absorbent polymer present region also may be disposed in the shape of a curved line extending in the longitudinal direction y, although not shown in the drawings. However, in the light of easily manufacturing the sheet-shaped absorbent body, the absorbent polymer present region is preferably disposed in the shape of a practically straight line extending in the longitudinal direction y.

In FIGS. 3B to 3D, the rectangular absorbent polymer present regions are disposed intermittently in both the width direction x and the longitudinal direction y. In FIG. 3B, the rectangular absorbent polymer present regions are disposed more densely in the longitudinal direction y than in the width direction x. In FIGS. 3C and 3D, the rectangular absorbent polymer present regions which are long in the longitudinal direction y are disposed intermittently in both the width direction x and the longitudinal direction y. When the absorbent polymer present regions are disposed in the manners shown in FIGS. 3B to 3D, a bodily fluid easily spreads in the longitudinal direction y. The absorbent polymer present regions are preferably aligned at least in the longitudinal direction y, and as a result, a bodily fluid easily spreads in the longitudinal direction y. For example, in FIGS. 3B and 3C, the rectangular absorbent polymer present regions are aligned in both the width direction x and the longitudinal direction y. In FIG. 3D, the rectangular absorbent polymer present regions are aligned only in the longitudinal direction y.

In FIGS. 3B to 3D, the shapes of the absorbent polymer present regions disposed intermittently in the width direction x and the longitudinal direction y are rectangular; however, the shapes of the absorbent polymer present regions may be, for example, elliptic which is long in the longitudinal direction y as shown in FIG. 3E. Further, the absorbent polymer present region also may have a circular shape, a rectangular shape whose corners are rounded, or the like, although not shown in the drawings.

Preferably, in the first absorbent layer and the second absorbent layer, each of the absorbent polymer present regions is disposed in the shape of a practically straight line extending in the longitudinal direction and having a length of 75% or more of the absorbent laminate in the longitudinal direction, and the absorbent polymer present regions are aligned practically parallel each other in the width direction of the absorbent laminate. When the absorbent polymer present regions are disposed in this manner, a bodily fluid easily spreads in the longitudinal direction y in the sheet-shaped absorbent body, and further, the absorptive capacity of the sheet-shaped absorbent body is easily enhanced since the absorbent polymer present regions are disposed so as to have relatively large areas. In addition, when the sheet-shaped absorbent body is continuous-manufactured, it becomes easy to form the absorbent polymer present region by applying an absorbent polymer on a nonwoven fabric. The length of the linear shaped absorbent polymer present region is preferably 80% or more of the length of the absorbent laminate in the longitudinal direction, more preferably 90% or more of the length of the absorbent laminate in the longitudinal direction, and further more preferably practically equal to the length of the absorbent laminate in the longitudinal direction.

In both the first absorbent layer and the second absorbent layer, in the case that each of the absorbent polymer present regions is disposed in the shape of a practically straight line extending in the longitudinal direction and having a length of 75% or more of the absorbent laminate in the longitudinal direction, and the absorbent polymer present regions are aligned practically parallel each other in the width direction of the absorbent laminate, it is preferable that a maximum distance between the adjacent absorbent polymer present regions of the first absorbent layer is larger than that of the second absorbent layer. In the sheet-shaped absorbent body, the larger the distances between the absorbent polymer present regions are, that is, the larger the widths of the absorbent polymer absent regions are, the more easily a bodily fluid in the sheet-shaped absorbent body is permeated and spread. For enhancing the permeation and spread of a bodily fluid in the sheet-shaped absorbent body, it is more effective to dispose a few absorbent polymer absent regions having wide widths, than to dispose many absorbent polymer absent regions having narrow widths. Therefore, when the maximum distance between the adjacent absorbent polymer present regions of the first absorbent layer is larger than that of the second absorbent layer, the absorbent laminate can absorb a bodily fluid more rapidly.

An example of the combination of the first absorbent layer and the second absorbent layer is the combination of a sheet-shaped absorbent body having a pattern as shown in FIG. 2A as the first absorbent layer and a sheet-shaped absorbent body having a pattern as shown in FIG. 2B as the second absorbent layer. Here, FIGS. 2A and 2B are assumed to have the same reduced scale. In FIG. 2A, the distances between the adjacent absorbent polymer present regions are the same and have a length h. In FIG. 2B, some of the distances between the adjacent absorbent polymer present regions are larger than the others thereof, and the larger distances, that is the maximum distance, have a length k. In FIGS. 2A and 2B, the maximum distance h between the adjacent absorbent polymer present regions of the first absorbent layer is larger than the maximum distance k between the adjacent absorbent polymer present regions of the second absorbent layer.

The absorbent laminate is formed by stacking the first absorbent layer on the second absorbent layer. The first absorbent layer and the second absorbent layer may be joined together by an adhesive or the like and fixed, in order that the absorbent laminate is suppressed to twist or deform in using the absorbent article. Thus, an adhesive layer may be provided between the first absorbent layer and the second absorbent layer. In this case, in order that a bodily fluid smoothly transfers to a lower layer, each adhesive layer is preferably formed into a net-like structure. As an adhesive used for these adhesive layers, the adhesive which can be used in the sheet-shaped absorbent body may be used.

The absorbent laminate may comprise another absorbent layer in addition to the first absorbent layer and the second absorbent layer. In this case, the another absorbent layer is preferably provided under the second absorbent layer, that is the back sheet side. Example of the another absorbent layer includes a layer which can be used for the second absorbent layer.

The absorbent article is preferably provided with a pair of rising flaps on both sides in the width direction. Providing the rising flaps enables to prevent lateral leakage of excretion such as urine. The rising flap may be formed, for example, by joining side sheets which extend in the longitudinal direction to the top sheet on the opposite sides in the width direction, and providing elastic members to inner ends, with respect to the width direction, of the side sheets. When the side sheet and the elastic member are provided in this manner, the inner ends of the side sheets rise toward a wearer due to a shrinkage force of the elastic member to form the rising flaps. The rising flap or the side sheet is preferably made of a liquid-impermeable plastic film, a liquid-impermeable nonwoven fabric, or the like.

Elastic materials such as a polyurethane thread, a polyurethane film, a natural rubber, which are generally used for absorbent articles such as a disposable diaper, can be used for the elastic member. The elastic member is preferably fixed in a stretched state with a hot-melt adhesive. For example, a polyurethane thread having a fineness of 100 dtex to 2,500 dtex is stretched at a ratio of 1.1 to 5.0 times to be fixed. A preferable bonding means is a rubber hot-melt adhesive.

The absorbent article of the present invention can be applied to an incontinence pad, a sanitary napkin, a disposable diaper, or the like. In the case that the absorbent article is a sanitary napkin, the absorbent core is disposed between the top sheet and the back sheet, thereby forming a sanitary napkin, for example. Examples of the shape of the sanitary napkin include a substantially rectangular shape, an hourglass shape and a center nipped-in gourd shape. In the case that the absorbent article is a disposable diaper, the disposable diaper may be an open-type disposable diaper which is provided with a pair of fastening members on left and right sides of a back part or a front part and which is formed into a pants shape by using the fastening members when being worn, or the disposable diaper may be a pants-type disposable diaper in which a front part and a back part are joined to each other to form a waist opening and a pair of leg openings.

The absorbent article of the present invention is explained in the following, referring to FIGS. 4 to 6, in which an incontinence pad is shown as an example (a first embodiment). FIG. 4 shows a plan view of an incontinence pad, which is one embodiment of the absorbent article of the present invention. FIG. 5 shows a cross-sectional view taken along line V-V of the incontinence pad shown in FIG. 4. FIG. 6 shows a cross-sectional view taken along line VI-VI of the incontinence pad shown in FIG. 4. In the drawings, an arrow x represents the width direction and an arrow y represents the longitudinal direction. A direction on the plane formed by the arrows x and y is defined as the planar direction, and a direction orthogonal to the arrows x and y is defined as a thickness direction or a vertical direction.

An absorbent article 1 comprises a top sheet 2, a back sheet 3 and an absorbent laminate 4 disposed between the top sheet 2 and the back sheet 3. A diffusion sheet 5 is provided between the top sheet 2 and the absorbent laminate 4, and a base sheet 6 is provided between the back sheet 3 and the absorbent laminate 4. However, in FIG. 4, the absorbent article is represented such that the diffusion sheet 5 is omitted.

The top sheet 2 is placed so as to face to a wearer's skin, and allows a bodily fluid such as urine to permeate through. The bodily fluid which has passed through the top sheet 2 diffuses in the planar direction through the diffusion sheet 5, and then transfers to the absorbent laminate 4. The diffusion sheet 5 is preferably liquid-permeable, and a material which can be used for the top sheet may be used as the diffusion sheet 5. However, the diffusion sheet 5 may not be provided.

The base sheet 6 may be liquid-permeable or liquid-impermeable. As the base sheet 6, a material which can be used for the top sheet or the back sheet may be used, or a heavyweight crepe paper (for example, a mass per unit area thereof is in the range of 30 g/m² to 50 g/m²). Providing the base sheet 6 gives shape retaining effect, a bodily fluid diffusion effect or the like to the absorbent article 1. However, the base sheet 6 may not be provided.

Side sheets 7, which extend in the longitudinal direction y, are provided to the top sheet 2 on both sides in the width direction x. The side sheet 7 is joined to the top sheet 2 at a joining portion 8. Three rising elastic members 9 are disposed at an inner end in the width direction x of the each side sheet 7. When the disposable diaper 1 is worn, the inner end of the side sheet 7 rises toward a wearer's skin due to a shrinkage force of the rising elastic members 9, thereby preventing excrement such as urine from leaking.

The absorbent laminate 4 has the longitudinal direction y and the width direction x, and comprises a first absorbent layer 11 and a second absorbent layer 21 provided in this order from the top sheet 2 side. Therefore, a bodily fluid which has transferred to the absorbent laminate 4 is basically first absorbed by the first absorbent layer 11. The first absorbent layer 11 and the second absorbent layer 21 are joined together by an adhesive layer 10.

The first absorbent layer 11 contains an absorbent polymer 14 but does not contain a pulp fiber between nonwoven fabric sheets 12, 13. In detail, the first absorbent layer 11 has a plurality of absorbent polymer present regions 15, in each of which the absorbent polymer 14 is provided, and absorbent polymer absent regions 16 adjacent to the absorbent polymer present region 15 between the nonwoven fabric sheets 12, 13; and the nonwoven fabric sheets 12, 13 are joined together at the absorbent polymer absent regions 16 to form sealing portions 17. The absorbent polymer 14 disposed at the absorbent polymer present region 15 is fixed to the nonwoven fabric sheets 12, 13 by the adhesive layer; and the nonwoven fabric sheets 12, 13 are joined together at the absorbent polymer absent regions 16 by the adhesive layer.

The second absorbent layer 21 contains an absorbent polymer 24 but does not contain a pulp fiber between nonwoven fabric sheets 22, 23. In detail, the second absorbent layer 21 has a plurality of absorbent polymer present regions 25, in each of which the absorbent polymer 24 is provided, and absorbent polymer absent regions 26 adjacent to the absorbent polymer present region 25 between the nonwoven fabric sheets 22, 23; and the nonwoven fabric sheets 22, 23 are joined together at the absorbent polymer absent regions 26 to form sealing portions 27. The absorbent polymer 24 disposed at the absorbent polymer present region 25 is fixed to the nonwoven fabric sheet 22, 23 by the adhesive layer; and the nonwoven fabric sheets 22, 23 are joined together at the absorbent polymer absent regions 26 by the adhesive layer.

The second absorbent layer 21 extends outward in the longitudinal direction y beyond the first absorbent layer 11, as shown in FIGS. 4 and 6. In the first absorbent layer 11, when a bodily fluid such as urine is excreted in a large amount at one time, the bodily fluid spreads in the longitudinal direction y, and a part of the body fluid may overflow from the surface of the first absorbent layer. In such a case, in the absorbent article 1, since the second absorbent layer 21 extends outward in the longitudinal direction y beyond the first absorbent layer 11, the bodily fluid which has overflowed from the edge of the first absorbent layer 11 in the longitudinal direction y can be absorbed by the second absorbent layer 21, and therefore, the low absorption rate of the first absorbent layer 11 is recovered.

In the first absorbent layer 11, the absorbent polymer present region 15 and the absorbent polymer absent region 16 are arranged in a pattern shown in FIG. 2A in a planar view. In the second absorbent layer 21, the absorbent polymer present region 25 and the absorbent polymer absent region 26 are arranged in a pattern shown in FIG. 2B in a planar view. That is, the absorbent polymer absent regions 16 of the first absorbent layer 11 are disposed intermittently in the width direction x of the absorbent laminate 4, and absorbent polymer absent regions 26 of the second absorbent layer 21 are disposed intermittently in the width direction x of the absorbent laminate 4. Each of the absorbent polymer present regions 15, 25 is disposed in a shape of a practically straight line extending in the longitudinal direction y of the absorbent laminate 4 and having a length approximately equal to the length of the first or second absorbent layer 11, 21 in the longitudinal direction y. The absorbent polymer present regions 15, 25 are aligned practically parallel each other in the width direction x of the absorbent laminate 4. When the absorbent polymer present regions 15, 25 and the absorbent polymer absent regions 16, 26 are disposed in this manner, a bodily fluid easily spreads in the longitudinal direction y, and as a result, a bodily fluid comes to be easily absorbed by the second absorbent layer 21.

In the first absorbent layer 11 and the second absorbent layer 21, the nonwoven fabric sheets are partly heat-sealed at the absorbent polymer absent regions 16, 26 to form heat-sealed portions 20, 30, as shown in partially enlarged views in FIGS. 2A and 2B. Therefore, a bodily fluid easily spreads in the planar direction at the heat-sealed portion 20, 30. In FIGS. 2A and 2B, each of the heat-sealed portions 20, 30 has a rhombic shape (or a partially lacked rhombic shape), and the rhombic-shaped heat-sealed portions 20, 30 are arranged in a matrix in a plane.

A maximum distance h between the adjacent absorbent polymer present regions 15 of the first absorbent layer 11 is larger than a maximum distance k between the adjacent absorbent polymer present regions 25 of the second absorbent layer 21. Therefore, permeation and spread of a bodily fluid in the first absorbent layer 11 are enhanced more, whereby the bodily fluid is more rapidly absorbed by the absorbent laminate 4.

The absorbent article of the present invention is explained referring to FIGS. 7 to 9, in which an incontinence pad is shown as another example (a second embodiment). FIG. 7 shows a plan view of an incontinence pad, which is another embodiment of the absorbent article of the present invention. FIG. 8 shows a cross-sectional view taken along line VIII-VIII of the incontinence pad shown in FIG. 7. FIG. 9 shows a cross-sectional view taken along line IX-IX of the incontinence pad shown in FIG. 7. In the following, the description of parts overlapping the first embodiment is omitted, and the reference number “21” of the second absorbent layer in the first embodiment is replaced by “31”.

An absorbent article 1 comprises a top sheet 2, a back sheet 3 and an absorbent laminate 4 disposed between the top sheet 2 and the back sheet 3. A diffusion sheet 5 is provided between the top sheet 2 and the absorbent laminate 4. The absorbent laminate 4 has the longitudinal direction y and the width direction x, and comprises a first absorbent layer 11 and a second absorbent layer 31 provided in this order from the top sheet 2 side. The first absorbent layer 11 is the same as in the first embodiment. In FIG. 7, the absorbent article is represented such that the diffusion sheet 5 is omitted.

The second absorbent layer 31 contains an absorbent polymer 32 and pulp fibers 33. The second absorbent layer 31 extends outward in the longitudinal direction y beyond the first absorbent layer 11 also in the second embodiment, as shown in FIGS. 7 and 9. Moreover, the second absorbent layer 31 extends outward in the width direction x beyond the first absorbent layer 11, as shown in FIGS. 7 and 8. In the second embodiment, since the second absorbent layer 31 contains pulp fibers 33, a bodily fluid which has overflowed from the first absorbent layer 11 is absorbed more rapidly by the second absorbent layer 31. In addition, the second absorbent layer 31 can receives a bodily fluid which has overflowed from the first absorbent layer 11 in both the longitudinal direction y and the width direction x.

The absorbent article of the present invention is explained referring to FIGS. 10 and 11, in which an incontinence pad is shown as still another example (a third embodiment). FIG. 10 shows a plan view of an incontinence pad, which is still another embodiment of the absorbent article of the present invention. FIG. 11 shows a cross-sectional view taken along line XI-XI of the incontinence pad shown in FIG. 10. In the following, the description of parts overlapping the first embodiment is omitted.

In the third embodiment, the length of the first absorbent layer 11 in the longitudinal direction y is shorter than that in the first embodiment. The absorbent article 1 is folded at a line a-a and a line b-b which extend in the width direction x, thereby folded in three. The first absorbent layer 11 is disposed between the line a-a and the line b-b, and does not cross neither the line a-a nor the line b-b. When the first absorbent layer is provided in this manner, the threefold absorbent article 11, which is obtained by folding the absorbent article in three, can be formed slimly. Therefore, such absorbent article 1 is convenient to be carried.

### REFERENCE SIGNS LIST

1: an absorbent article (an incontinence pad)

2: a top sheet

3: a back sheet

4: an absorbent laminate

11: a first absorbent layer

21, 31: a second absorbent layer

12, 13, 22, 23: a nonwoven fabric sheet

14, 24, 32: an absorbent polymer

15, 25: an absorbent polymer present region

16, 26: an absorbent polymer absent region

33: pulp fibers

1. An absorbent article comprising a top sheet, a back sheet and an absorbent laminate disposed between the top sheet and the back sheet, wherein:
the absorbent laminate comprises a first absorbent layer and a second absorbent layer provided in this order from the top sheet side, and having a longitudinal direction and a width direction;
the second absorbent layer contains an absorbent polymer and/or pulp fibers;
the first absorbent layer contains an absorbent polymer but does not contains a pulp fiber between nonwoven fabric sheets; and
the second absorbent layer extends outward in the longitudinal direction beyond the first absorbent layer.

2. The absorbent article according to claim 1, wherein:
the first absorbent layer has a plurality of absorbent polymer present regions, in each of which the absorbent polymer is provided, and an absorbent polymer absent region adjacent to the absorbent polymer present region between the nonwoven fabric sheets;
the nonwoven fabric sheets of the first absorbent layer are joined together at the absorbent polymer absent region to form a sealing portion; and
the absorbent polymer present regions of the first absorbent layer are disposed intermittently in the width direction of the absorbent laminate.

3. The absorbent article according to claim 2, wherein the second absorbent layer contains an absorbent polymer but does not contains a pulp fiber between nonwoven fabric sheets.

4. The absorbent article according to claim 3, wherein:
the second absorbent layer has a plurality of absorbent polymer present regions, in each of which the absorbent polymer is provided, and an absorbent polymer absent region adjacent to the absorbent polymer present region between the nonwoven fabric sheets;
the nonwoven fabric sheets of the second absorbent layer are joined together at the absorbent polymer absent region to form a sealing portion; and
the absorbent polymer present regions of the second absorbent layer are disposed intermittently in the width direction of the absorbent laminate.

5. The absorbent article according to claim 4, wherein the nonwoven fabric sheets of at least one of the first absorbent layer and the second absorbent layer are partly heat-sealed at the absorbent polymer absent region.

6. The absorbent article according to claim 4, wherein
each of the absorbent polymer present regions is disposed in a shape of a practically straight line extending in the longitudinal direction and having a length of 75% or more of the absorbent laminate in the longitudinal direction; and
the absorbent polymer present regions are aligned practically parallel each other in the width direction of the absorbent laminate.

7. The absorbent article according to claim 6, wherein a maximum distance between the adjacent absorbent polymer present regions of the first absorbent layer is larger than that of the second absorbent layer.

8. The absorbent article according to claim 4, wherein the nonwoven fabric sheets of the first absorbent layer or the second absorbent layer are kept joined together when the first absorbent layer or the second absorbent layer absorbs a body fluid.

9. The absorbent article according to claim 4, wherein:
an adhesive is applied to the nonwoven fabric sheet to form an adhesive layer;
the absorbent polymer disposed at the absorbent polymer present region is fixed to the nonwoven fabric sheet by the adhesive layer; and
the nonwoven fabric sheets are joined together at the absorbent polymer absent region by the adhesive layer.

10. The absorbent article according to claim 1, wherein:
the absorbent article is folded at one or more fold line(s) extending in the width direction; and
the first absorbent layer is provided so as not to cross at least one of the fold line(s).

11. The absorbent article according to claim 2, wherein the nonwoven fabric sheets of the first absorbent layer are partly heat-sealed at the absorbent polymer absent region.

12. The absorbent article according to claim 2, wherein
each of the absorbent polymer present regions is disposed in a shape of a practically straight line extending in the longitudinal direction and having a length of 75% or more of the absorbent laminate in the longitudinal direction; and
the absorbent polymer present regions are aligned practically parallel each other in the width direction of the absorbent laminate.

13. The absorbent article according to claim 2, wherein the nonwoven fabric sheets of the first absorbent layer are kept joined together when the first absorbent layer or the second absorbent layer absorbs a body fluid.

14. The absorbent article according to claim 2, wherein:
an adhesive is applied to the nonwoven fabric sheet to form an adhesive layer;
the absorbent polymer disposed at the absorbent polymer present region is fixed to the nonwoven fabric sheet by the adhesive layer; and
the nonwoven fabric sheets are joined together at the absorbent polymer absent region by the adhesive layer.

15. The absorbent article according to claim 2, wherein:
the absorbent article is folded at one or more fold line(s) extending in the width direction; and
the first absorbent layer is provided so as not to cross at least one of the fold line(s).

16. The absorbent article according to claim 4, wherein:
the absorbent article is folded at one or more fold line(s) extending in the width direction; and
the first absorbent layer is provided so as not to cross at least one of the fold line(s).
